(19) United States
(12) Patent Application Publication
(10) Pub. No.: US 2004/0185024 A1
(43) Pub. Date: Sep. 23, 2004

(54) Composition for washing keratin materials comprising at least one amphoteric surfactant and at least one polyethylene glycol ester or ether comprising at least one hydrophobic group

(76) Inventor: Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/737,789

(22) Filed: Dec. 18, 2003

Related U.S. Application Data
(60) Provisional application No. 60/439,445, filed on Jan. 13, 2003.

(30) Foreign Application Priority Data
Dec. 19, 2002 (FR) 02 16198

(51) Int. Cl. A61K 7/075; A61K 7/08

(52) U.S. Cl. 424/70.21

(57) ABSTRACT

The invention relates to cosmetic compositions containing, in a cosmetically acceptable medium, at least one amphoteric surfactant chosen from alkylamphohydroxyalkylsulphonates and salts thereof, and at least one polyethylene glycol ester or ether containing at least one C₈-C₄₀ hydrophobic group. These compositions are used for example for washing keratin materials, such as hair or skin.

This application claims benefit of U.S. Provisional Application No. 60/439,445, filed Jan. 13, 2003.

Disclosed herein is a composition for washing keratin materials, such as hair and/or skin, comprising at least one amphoteric surfactant chosen from alkylamphohydroxyalkylsulphonate compounds and salts thereof and at least one polyethylene glycol derivative comprising at least one hydrophobic group, and a washing process using the disclosed composition.

It is known to use nonionic surfactants, such as polyoxyethylenated esters, in shampoos. These PEG esters may be used as thickeners in this type of formulation.

European Patent No. EP 900 261 describes detergent and shampoo compositions based on combinations of ethoxylated esters that are only very mildly irritant to the eyes and that apparently have satisfactory foaming power. These detergent compositions comprise a hydrophobic polyoxyethylenic derivative as a nonionic surfactant, an anionic surfactant, and betaine as a third surfactant. It is the combination of these three compounds that is said to give the composition a foam with good stability that is only mildly irritating to the eyes.

Alkylamphohydroxyalkylsulphonate amphoteric surfactants have already been recommended in detergent cosmetic compositions. They have been described for example, in patent application WO 99/36054.

However, these known compositions may not always have satisfactory cosmetic properties.

Disclosed herein is thus a detergent cosmetic composition that may have at least one advantageous cosmetic property chosen from, for example, improved disentangling of the hair, improved smoothing of the hair, and the ability to give the hair flexibility, manageability, lightness, volume, and softness.

The present inventors have found, surprisingly, that it is possible to formulate a cosmetic composition as disclosed herein, for example a composition for washing keratin materials, such as shampoos with, for example, good ocular tolerance and/or improved cosmetic properties. The cosmetic composition comprises at least one polyethylene glycol ester or ether comprising at least one hydrophobic group and at least one amphoteric surfactant chosen from alkylamphohydroxyalkylsulphonate compounds and salts thereof.

Accordingly, disclosed herein is a composition for washing keratin materials, comprising at least one amphoteric surfactant chosen from alkylamphohydroxyalkylsulphonate compounds and salts thereof and at least one polyethylene glycol ester or ether comprising at least one C₈-C₄₀ hydrophobic group.

Also disclosed herein is the use of a composition as described above to improve at least one property chosen from disentangling the hair, and smoothing the hair, and to give hair at least one of volume, lightness, softness, flexibility, and manageability.

Disclosed herein is a process for treating keratin materials, such as hair, comprising applying a cosmetic composition as disclosed herein to the keratin materials. For example, the process may be a process for washing keratin materials.

Other subjects of the invention will become apparent on reading the description and the examples that follow.

As used herein, the term “keratin materials” means at least one material chosen from, for example, the hair, the eyelashes, the eyebrows, the skin, the nails, mucous membranes, and the scalp. In one embodiment, the keratin material used herein is hair. As used herein, the term “keratin fibers” means at least one fiber chosen from, for example, the hair, the eyelashes, and the eyebrows. In one embodiment, the keratin fibers used herein are hair.

As used herein, the term “hydrophobic group” means a group containing from 8 to 40 carbon atoms.

The at least one alkylamphohydroxyalkylsulphonate amphoteric surfactants and salts thereof may be of formula (I) below:

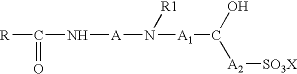

wherein:

R is chosen from saturated and unsaturated, linear and branched hydrocarbon-based radicals comprising from 5 to 29 carbon atoms,

R, for example, may be chosen from mono- and polyunsaturated alkyl and alkenyl radicals comprising from 5 to 29 carbon atoms, for example from 7 to 22 carbon atoms, and as a further example from 9 to 17 carbon atoms,

R1 is a C₁-C₄ hydroxyalkyl radical, for example hydroxyethyl,

A, A₁, and A₂, which may be identical or different, are chosen from linear and branched C₁-C₁₀ divalent alkylene radicals, and for example from linear and branched C₁-C₃ divalent alkylene radicals, and

X is chosen from a hydrogen atom, a mineral cation, and an organic cation chosen from that of an alkali metal (for example Na⁺ or K⁺), that of an alkaline-earth metal (Mg²⁺ or Ca²⁺), an NH₄⁺ ion, and ammonium ions derived from basic amino acids or from amino alcohols.

For example, the at least one amphoteric surfactant disclosed herein may be a compound of formula (I) wherein R is chosen from saturated, linear and branched alkyl radicals containing from 7 to 29 carbon atoms, for example from 7 to 22 carbon atoms.

When X is an ammonium ion derived from an alkanolamine, this alkanolamine may be chosen from monoethanolamine, diethanolamine, triethanolamine, and 3-amino-1,2-propanediol. When X is an ammonium ion derived from an amine, this amine may be a basic amino acid chosen from, for example, lysine, arginine, sarcosine, ornithine, and citrulline.

For example, in one embodiment, A may be identical to A₂ and be —CH₂CH₂—, A₁ may be —CH₂—, and X may be Na⁺.

The at least one amphoteric surfactant of formula (I) may, for example, be chosen from cocoyl amphohydroxypropyl sulphonate salts, for example the sodium salt, such as the product sold under the name MIRANOL CSE by the company Rhodia Chimie, or palmitoyl amphohydroxypropyl sulphonate salts.

The at least one amphoteric surfactant chosen from alkylamphohydroxyalkyl-sulphonate compounds and salts thereof may be present in the composition disclosed herein in an amount ranging from 0.1% to 30% by weight, for example from 1% to 20% by weight, and as a further example from 1.5% to 15% by weight relative to the total weight of the final composition.

The at least one polyethylene glycol ester or ether comprising at least one hydrophobic group is chosen, for example, from the compounds of formulae (II) and (III) below:

R₂—(O—CH₂—CH₂)n—OR₃ (II)

R₂—(O—CH₂—CH₂)m—OH (III)

wherein:

R₂ is chosen from linear and branched, saturated and unsaturated C₈-C₄₀ alkyl, C₈-C₄₀ acyl, alkylaryl, and aralkyl groups comprising from 8 to 40 carbon atoms, a glyceryl group, a (C₈-C₄₀)alkylglyceryl group, an acyl group chosen from lanolin oil and lanolin fat, coconut oil, jojoba oil, castor oil, sweet almond oil, groundnut oil, wheatgerm oil, rice bran oil, flax oil, apricot kernel oil, walnut oil, palm oil, pistachio oil, sesame seed oil, rapeseed oil, cade oil, maize oil, peach nut oil, poppy oil, pine oil, soybean oil, avocado oil, sunflower seed oil, hazelnut oil, olive oil, grapeseed oil, safflower oil, shea butter, and babassu oil,

R₃ is chosen from a hydrogen atom, and linear and branched, saturated and unsaturated alkyl and acyl groups comprising from 1 to 40 carbon atoms, and n and m are integers ranging from 3 to 350.

For example, R₂ may be an acyl group containing from 12 to 20 carbon atoms, R₃ may be an acyl group containing from 12 to 20 carbon atoms, and n may be an integer ranging from 100 to 300.

In one embodiment, the weight ratio of the hydrophilic portion (—(O—CH₂—CH₂)nO) to the hydrophobic portion (R₂ and/or R₃) may range from 8 to 1,000.

A compound of formula (II) may be used, for example, wherein R₂ and R₃ are both chosen from an acyl group comprising from 12 to 20 carbon atoms and wherein n ranges from 100 to 300. Examples that may be mentioned include PEG 150 distearate and PEG 250 distearate. Such compounds are sold under the name EMANON 3299R by the company KAO and under the name KESSCO PEG 6000 DS by the company Akzo.

As disclosed herein, it may also be possible to use at least one polyethylene glycol ester or ether of formula (IV)

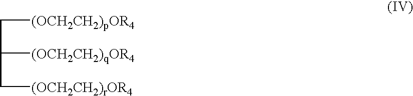

wherein R₄ is chosen from a hydrogen atom, and linear and branched, saturated and unsaturated C₈-C₄₀ alkyl, C₈-C₄₀ acyl, alkylaryl, and aralkyl groups comprising from 8 to 40 carbon atoms, and

p, q and r, which may be identical or different, range from 0 to 350,

the sum p+q+r ranges from 5 to 350, and

with the proviso that at least one of the groups R₄ is other than a hydrogen atom.

The groups R₄ may be, for example, acyl groups derived from plant oils such as coconut oil, jojoba oil, castor oil, sweet almond oil, groundnut oil, wheatgerm oil, rice bran oil, flax oil, apricot kernel oil, walnut oil, palm oil, pistachio oil, sesame seed oil, rapeseed oil, cade oil, maize oil, peach nut oil, poppy oil, pine oil, soybean oil, avocado oil, sunflower seed oil, hazelnut oil, olive oil, grapeseed oil, safflower oil, shea butter, and babassu oil.

Palm oil glycerides oxyethylenated with 200 mol of ethylene oxide, coconut oil mono-, di- and triglycerides oxyethylenated with 30 mol of ethylene oxide, and coconut oil mono-, di- and triglycerides oxyethylenated with 7 mol of ethylene oxide may, for example, be used.

According to the present disclosure, the at least one polyethylene glycol ester or ether may be present in the cosmetic composition in an amount ranging from 0.1% to 20%, and for example from 2% to 15% by weight relative to the total weight of the composition.

The composition disclosed herein may also comprise at least one other or additional surfactant which may be present in an amount ranging from 0.1% to 60% by weight, relative to the total weight of the composition, for example ranging from 0.5% to 30%, relative to the total weight of the composition and as a further example ranging from 3% to 15%, relative to the total weight of the composition.

The at least one other surfactant may be chosen from anionic, amphoteric, nonionic, and cationic surfactants, provided it is different from the at least one amphoteric surfactant described above.

The at least one other or additional surfactant that may be used, is chosen from, for example:

(i) Anionic surfactant(s):

In the context of the present disclosure, their nature is not a truly critical feature.

As examples of anionic surfactants which can be used herein, alone or as mixtures, mention may be made for example (non-limiting list) of salts (for example alkali metal salts, such as sodium salts, ammonium salts, amine salts, amino alcohol salts or magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates; and N-acyltaurates. The alkyl or acyl radical(s) of all of these various compounds, for example, comprises from 8 to 24 carbon atoms. The aryl radical for example, may be chosen from a phenyl and a benzyl group.

Among the anionic surfactants which may be used, non-limiting mention may also be made of fatty acid salts such as the salts of oleic, ricinoleic, palmitic, and stearic acids, coconut oil acid and hydrogenated coconut oil acid; and acyl lactylates wherein the acyl radical contains 8 to 20 carbon atoms. Weakly anionic surfactants may also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example those comprising from 2 to 50 ethylene oxide groups, and mixtures thereof.

An anionic surfactant chosen from sodium, magnesium or ammonium (C₁₂-C₁₄)alkyl sulphates, sodium, magnesium, triethanolamine or ammonium (C₁₂-C₁₄)alkyl ether sulphates oxyethylenated with 2 to 5 mol of ethylene oxide (such as from 2 to 3 mol), sodium cocoyl isethionate and mixtures thereof, may, for example, also be used.

Among the anionic surfactants, alkyl sulphate salts, alkyl ether sulphate salts, and mixtures thereof may, for example, be used.

(ii) Non-ionic surfactant(s):

The non-ionic surfactants are also compounds that are well known per se (see for example in this respect M. R. Porter, “Handbook of Surfactants,” Blackie & Son (Glasgow and London), pp.116-178(1991) and, as used herein, their nature is not a critical feature. Thus, they can be selected for example from (non-limiting list) polyethoxylated and polypropoxylated alkylphenols, alpha-diols and alcohols having a fatty chain comprising, for example, 8 to 18 carbon atoms, it being possible for the number of ethylene oxide or propylene oxide groups to range, for example, from 2 to 50. Non-limiting mention may also be made of copolymers of ethylene oxide and of propylene oxide, condensates of ethylene oxide and of propylene oxide with fatty alcohols; polyethoxylated fatty amides having, for example, from 2 to 30 mol of ethylene oxide, polyglycerolated fatty amides containing on average 1 to 5, and for example 1.5 to 4, glycerol groups; oxyethylenated fatty acid esters of sorbitan having from 2 to 30 mol of ethylene oxide; fatty acid esters of sucrose, fatty acid esters of polyethylene glycol, alkyl polyglycosides, N-alkylglucamine derivatives, amine oxides such as (C₁₀-C₁₄)alkylamine oxides or N-acylaminopropylmorpholine oxides. Alkyl polyglycosides may be used as the non-ionic surfactants herein, for example.

(iii) Amphoteric surfactant(s):

The additional amphoteric surfactants, whose nature is not a critical feature herein, may be chosen from, for example, aliphatic secondary or tertiary amine derivatives wherein the aliphatic radical is a linear or branched chain comprising 8 to 18 carbon atoms and comprising at least one water-solubilizing anionic group (for example carboxylate, sulphonate, sulphate, phosphate or phosphonate). Non-limiting mention may also be made of (C₈-C₂₀)alkylbetaines, sulphobetaines, (C₈-C₂₀)alkyl-amido(C₁-C₆)alkylbetaines or (C₈-C₂₀)alkylamido(C₁-C₆)alkylsulphobetaines.

Among the amine derivatives, non-limiting mention may be made of the products sold under the name MIRANOL, as described in U.S. Pat. Nos. 2,528,378 and 2,781,354:

R₂—CONHCH₂CH₂—N(R₃)(R₄)(CH₂COO⁻)

wherein:

R₂ is an alkyl radical of an acid R₂—COOH present in hydrolyzed coconut oil, or is chosen from heptyl, nonyl, and undecyl radicals,

R₃ is a β-hydroxyethyl group, and R₄ is a carboxymethyl group; and

R₅—CONHCH₂CH₂—N(B)(D)

wherein:

B is —CH₂CH₂OX′,

D is —(CH₂)_{z}—Y′, wherein z is chosen from 1 and 2,

X′ is chosen from a —CH₂CH₂—COOH group, and a hydrogen atom,

Y′ is chosen from a —COOH radical and a —CH₂—CHOH—SO₃H radical,

R₅ is chosen from an alkyl radical of an acid R₉—COOH present in coconut oil or in hydrolysed linseed oil, an alkyl radical, for example a C₇, C₉, C₁₁ or C₁₃ alkyl radical, a C₁₇ alkyl radical and its iso form, and an unsaturated C₁₇ radical, and R₉ is chosen from saturated and unsaturated, linear and branched alkyl groups having from 8 to 22 carbon atoms, such as from 8 to 16 carbon atoms.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylampho-dipropionate, disodium capryloamphodipropionate, lauroamphodipropionic acid, and cocoamphodipropionic acid.

By way of example, mention may be made of the cocoamphodiacetate sold under the trade name MIRANOL® C2M concentrate by the company Rhodia Chimie.

In one embodiment, the composition disclosed herein comprises at least one additional surfactant chosen from anionic and nonionic surfactants.

(iv) Cationic surfactant(s):

The composition may also contain at least one cationic surfactant present in the composition in an amount ranging from 0.001% to 5% by weight relative to the total weight of the composition.

The cationic surfactants may be, for example, chosen from primary, secondary, and tertiary fatty amine salts, such as polyoxyalkylenated; quaternary ammonium salts; imidazoline derivatives; and amine oxides of cationic nature.

The quaternary ammonium salts that may be used are chosen from, for example, tetralkylammonium halides (for example chlorides), for example dialkyldimethylammonium or alkyltrimethylammonium chlorides, wherein the alkyl radical comprises from 12 to 22 carbon atoms, for example behenyltrimethylammonium chloride, distearyldimethylammonium chloride, cetyltrimethyl-ammonium chloride, benzyldimethylstearylammonium chloride and stearamidopropyldimethyl(myristyl acetate)ammonium chloride, sold under the name CEPHARYL 70″ by the company Van Dyk.

Diacyloxyethyldimethylammonium, diacyloxyethylhydroxyethyl-methylammonium, monoacyloxyethyldihydroxyethylmethylammonium, triacyloxyethylmethylammonium and monoacyloxyethylhydroxyethyldimethylammonium salts (for example chlorides or methyl sulphate) and mixtures thereof may also be used. The acyl radicals for example originate from a plant oil, such as palm oil or sunflower oil.

The total amount of surfactants may range from 3.5% to 55% by weight and for example from 7% to 30% by weight relative to the total weight of the composition.

The compositions disclosed herein may also contain silicones, i.e., polyorganosiloxane oils, or polyorganosiloxane gums or resins, such as in the form of solutions in organic solvents, emulsions, or microemulsions.

Among the polyorganosiloxanes that may be used in the composition disclosed herein, mention may be made in a non-limiting manner of:

volatile silicones: these have a boiling point ranging from 60° C. to 260° C. They may be chosen from cyclic silicones comprising from 3 to 7 and for example 4 to 5 silicon atoms, and

non-volatile silicones: they comprise mainly polyalkylsiloxanes, polyarylsiloxanes, polyalkylarylsiloxanes, silicone gums, silicone resins and mixtures thereof, and organomodified silicones.

Polydimethylsiloxanes, aminosilicones and oxyalkylenated silicones may be used, for example. The polyorganosiloxanes may be used in an amount ranging from 0.01% to 20% by weight and, for example, ranging from 0.1% to 10% by weight relative to the total weight of the composition.

The composition disclosed herein may also contain at least one cationic polymer.

As used herein, the term “cationic polymer” may be any polymer comprising cationic groups and/or groups that may be ionized into cationic groups.

The at least one cationic polymer that may be used in the composition disclosed herein may be chosen from all those already known per se as, for example, improving the cosmetic properties, for example those described in patent application EP-A-0 337 354 and in French patent applications FR-A-2 270 846, 2 383 660, 2 598 611, 2 470 596 and 2 519 863.

The at least one cationic polymer that may be used, for example, is chosen from those containing units comprising primary, secondary, tertiary, and quaternary amine groups that either may form part of the main polymer chain or may be borne by a side substituent directly attached thereto.

The at least one cationic polymer may have a number-average molecular mass ranging from 500 to 5×10⁶ approximately and, for example, ranging from 10³ to 3×10⁶ approximately.

Examples of the at least one cationic polymer that may be mentioned include polymers of the polyamine, polyamino amide, and polyquaternary ammonium type. These are known products.

The polymers of the polyamine, polyaminoamide and polyquaternary ammonium type that may be used in the composition disclosed herein, and that may for example, be mentioned, are those described in French patents Nos 2 505 348 and 2 542 997. Among these polymers, non-limiting mention may be made of:

(1) homopolymers or copolymers derived from acrylic or methacrylic esters or amides and comprising at least one of the units of the following formulae:

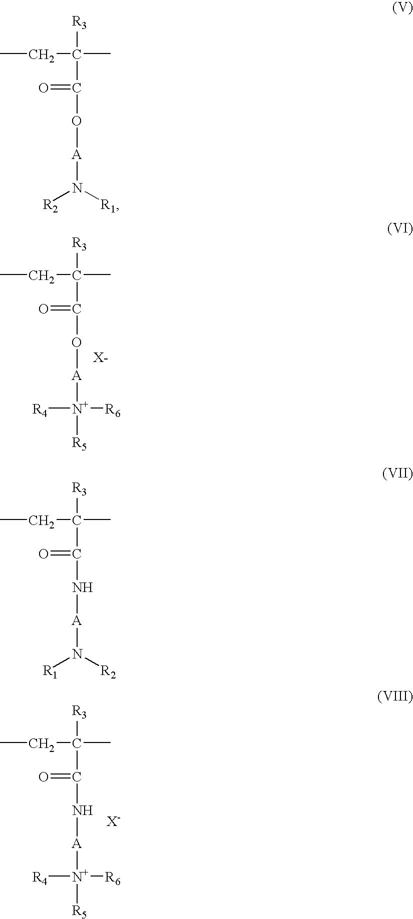

wherein:

R₁ and R₂, which may be identical or different, are chosen from hydrogen and C₁-C₆ alkyl groups, for example, methyl or ethyl;

R₃, which may be identical or different, is chosen from a hydrogen atom and a CH₃ radical;

A, which may be identical or different, is chosen from linear and branched alkyl groups of 1 to 6 carbon atoms, for example of 2 or 3 carbon atoms, or a hydroxyalkyl group of 1 to 4 carbon atoms;

R₄, R₅, R₆, which may be identical or different, are chosen from an alkyl group comprising from 1 to 18 carbon atoms, and a benzyl radical and for example an alkyl group comprising from 1 to 6 carbon atoms;

X⁻ is chosen from an anion derived from a mineral acid, and organic acid, such as a methyl sulphate anion, an ethyl sulphate anion or a halide such as chloride or bromide.

The copolymers of family (1) may also contain at least one unit derived from comonomers which may be chosen from the family of acrylamides, methacrylamides, diacetone acrylamides, acrylamides and methacrylamides substituted on the nitrogen with at least one group chosen from lower (C₁-C₄) alkyls, acrylic acids, methacrylic acids, acrylic esters, methacrylic esters , vinyllactams such as vinylpyrrolidone or vinylcaprolactam, and vinyl esters.

Thus, among these copolymers of family (1), non-limiting mention may be made of:

copolymers of acrylamide and of dimethylaminoethyl methacrylate quaternized with dimethyl sulphate or with a dimethyl halide, such as the product sold under the name HERCOFLOC by the company Hercules,

the copolymers of acrylamide and of methacryloyloxyethyl-trimethylammonium chloride described, for example, in patent application EP-A-080 976, and sold under the name BINA QUAT P 100 by the company Ciba,

the copolymer of acrylamide and of methacryloyloxyethyl-trimethylammonium methosulphate sold under the name RETEN by the company Hercules,

quaternized or nonquaternized vinylpyrrolidone/dialkylaminoalkyl acrylate or methacrylate copolymers, such as the products sold under the name GAFQUAT by the company ISP, such as, for example, GAFQUAT® 734 or GAFQUAT® 755, or alternatively the products known as COPOLYMER 845, 958 and 937. These polymers are described in detail in French patents 2 077 143 and 2 393 573,

dimethylaminoethyl methacrylate/vinylcaprolactam/vinylpyrrolidone terpolymers, such as the product sold under the name GAFFIX® VC 713 by the company ISP,

vinylpyrrolidone/methacrylamidopropyldimethylamine copolymers sold in particular under the name STYLEZE® CC 10 by ISP, and

quaternized vinylpyrrolidone/dimethylaminopropylmethacrylamide copolymers, such as the product sold under the name GAFQUAT® HS 100 by the company ISP.

(2) Cationic polysaccharides, for example, cationic celluloses, cationic starches and cationic galactomannan gums. Among the cationic polysaccharides that may be mentioned, for example, are cellulose ethers comprising quaternary ammonium groups, cationic cellulose copolymers or celluloses grafted with a water-soluble quaternary ammonium monomer and cationic galactomannan gums.

The cellulose ethers comprising quaternary ammonium groups, which are described in French patent 1 492 597, and for example the polymers sold under the names JR (JR 400, JR 125, JR 30M) or LR (LR 400, LR 30M) by the company Amerchol may, for example, be used. These polymers are also defined in the CTFA dictionary as hydroxyethylcellulose quaternary ammoniums that have reacted with an epoxide substituted with a trimethylammonium group.

The cationic cellulose copolymers or celluloses grafted with a water-soluble quaternary ammonium monomer are described, for example, in U.S. Pat. No. 4,131,576. Mention may be made, for example, of hydroxyalkylcelluloses, such as hydroxymethyl-, hydroxyethyl- and hydroxypropylcelluloses grafted for example with a methacryloylethyltrimethylammonium, methacrylamidopropyltrimethylammonium or dimethyldiallylammonium salt.

The commercial products corresponding to this definition are for example the products sold under the names CELQUAT® L 200 and CELQUAT® H 100 by the company National Starch.

The cationic galactomannan gums are described more particularly in U.S. Pat. Nos. 3,589,578 and 4,031,307, for example guar gums comprising trialkyl-ammonium cationic groups. Use may be made, for example, of guar gums modified with a salt (e.g. chloride) of 2,3-epoxypropyltrimethylammonium.

Such products are sold for example under the trade names JAGUAR® C13 S, JAGUAR® C 15, JAGUAR® C 17 or JAGUAR® C162 by the company Rhodia Chimie.

Starches modified with a 2,3-epoxypropyltrimethylammonium salt (e.g. chloride), such as the product known as Starch hydroxypropyltrimonium chloride according to the INCl nomenclature and sold under the name SENSOMER Cl-50 from Ondeo, may also used.

(3) Polymers comprising piperazinyl units and of divalent alkylene or hydroxyalkylene radicals containing straight or branched chains, optionally interrupted by oxygen, sulphur or nitrogen atoms or by aromatic or heterocyclic rings, as well as the oxidation and/or quaternization products of these polymers may also be used. Such polymers are described, for example, in French patents 2 162 025 and 2 280 361.

(4) Water-soluble polyamino amides prepared for example by polycondensation of an acidic compound with a polyamine. These polyamino amides may be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative, the crosslinking agent being used in proportions ranging from 0.025 to 0.35 mol per amine group of the polyamino amide. These polyamino amides may be alkylated or, if they contain at least one tertiary amine functions, they may be quaternized. Such polymers are described, for example, in French patents 2 252 840 and 2 368 508.

(5) Polyaminoamides resulting from the condensation of polyalkylene polyamines with polycarboxylic acids followed by alkylation with difunctional agents. Mention may be made, for example, of adipic acid/dialkylamino-hydroxyalkyldialkylenetriamine polymers wherein the alkyl radical contains from 1 to 4 carbon atoms and for example may be methyl, ethyl or propyl. Such polymers are described in particular in French patent 1 583 363.

Among these polyaminoamides, mention may be made for example of the adipic acid/dimethylaminohydroxypropyl/diethylenetriamine polymers sold under the name CARTARETINE® F, F4 or F8 by the company Sandoz.

(6) Polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms, the molar ratio of the polyalkylene polyamine to the dicarboxylic acid ranging from 0.8:1 to 1.4:1, and the polyamino amide resulting therefrom being reacted with epichlorohydrin in a molar ratio of epichlorohydrin relative to the secondary amine group of the polyamino amide ranging from 0.5:1 to 1.8:1. Such polymers are described in particular in U.S. Pat. Nos. 3,227,615 and 2,961,347.

Polymers of this type are sold in particular under the name HERCOSETT® 57 by the company Hercules Inc. or alternatively under the names PD 170 or DELSETTE® 101 by the company Hercules in the case of the adipic acid/epoxypropyl/diethylenetriamine copolymer.

(7) Cationic cyclopolymers such as alkyldiallylamine or dialkyldiallylammonium, such as the homopolymers or copolymers comprising, as the main constituent of the chain, units corresponding to formula (IX) or (X):

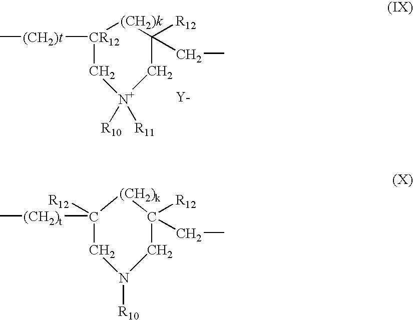

wherein k and t are equal to 0 or 1, the sum k+t being equal to 1; R₁₂ is chosen from a hydrogen atom and a methyl radical; R₁₀ and R₁₁, independently of each other, are chosen from an alkyl group having from 1 to 8 carbon atoms, a hydroxyalkyl group wherein the alkyl group for example has 1 to 5 carbon atoms, and a lower C₁-C₄ amidoalkyl group, or R₁₀ and R₁₁ can be, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate or phosphate. These polymers are described in particular in French patent 2 080 759 and in its Certificate of Addition 2 190 406.

In one embodiment, R₁₀ and R₁₁, independently of each other, may be an alkyl group comprising from 1 to 4 carbon atoms.

Among the polymers defined above, mention may be made for example of the dimethyldiallylammonium chloride homopolymer sold under the name MERQUAT® 100 by the company Nalco (and its homologues of low weight-average molecular mass) and copolymers of diallyidimethylammonium chloride and of acrylamide, sold under the name MERQUAT® 550.

(8) Quaternary diammonium polymers comprising repeating units corresponding to the formula:

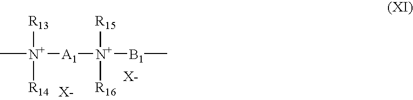

wherein: R₁₃, R₁₄, R₁₅ and R₁₆, which may be identical or different, are chosen from aliphatic, alicyclic, and arylaliphatic radicals comprising from 1 to 20 carbon atoms, and lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ are chosen from linear and branched C₁-C₆ alkyl radicals substituted with a nitrile, ester, acyl or amide group or a group —CO—O—R₁₇—D or —CO—NH—R₁₇—D where R₁₇ is an alkylene and D is a quaternary ammonium group;

A₁ and B₁, which may be identical or different, are chosen from linear and branched, saturated and unsaturated polymethylene groups comprising from 2 to 20 carbon atoms, and which may contain, linked to or intercalated in the main chain, at least one aromatic ring or at least one oxygen or sulphur atom or sulphoxide, sulphone, disulphide, amino, alkylamino, hydroxyl, quaternary ammonium, ureido, amide or ester groups, and

X⁻ is an anion derived from an inorganic or organic acid;

wherein A₁, R₁₃ and R₁₅ can form, with the two nitrogen atoms to which they are attached, a piperazine ring. If A₁ is chosen from linear and branched, saturated and unsaturated alkylene and hydroxyalkylene radicals, B₁ can also be a group (CH₂)n—CO—D—OC—(CH₂)n— wherein n is from 1 to 100 and for example from 1 to 50, and wherein D is:

a) a glycol residue of formula: —O—Z—O—, where Z is chosen from linear and branched hydrocarbon-based radicals or a group corresponding to one of the following formulae:

—(CH₂—CH₂—O)ₓ—CH₂—CH₂—

—[CH₂—CH(CH₃)—O]_{y}—CH₂—CH(CH₃)—

where x and y are an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y is chosen from linear and branched hydrocarbon-based radicals, or alternatively the divalent radical —CH₂—CH₂—S—S—CH₂—CH₂—; and

d) a ureylene group of formula: —NH—CO—NH—;

For example, X⁻ is a monovalent mineral or organic anion such as a halide (chloride or bromide), a sulphate or a carboxylate (acetate, lactate or citrate).

These polymers may have a number-average molecular mass ranging from 1,000 to 100,000.

Polymers of this type are described in particular in French patents 2 320 330, 2 270 846, 2 316 271, 2 336 434 and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

It is, for example, possible to use polymers that comprise repeating units corresponding to formula (XII):

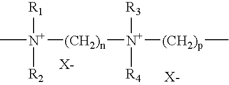

wherein: R₁, R₂, R₃ and R₄, which may be identical or different, are chosen from an alkyl and hydroxyalkyl radical comprising from 1 to 4 carbon atoms approximately, n and p are integers ranging from 2 to 20 approximately, and X⁻ is an anion derived from an inorganic or organic acid.

One compound of formula (XII) which may, for example, be used is the one for which R₁, R₂, R₃ and R₄ are a methyl radical, n=3, p=6, and X=Cl, which is known as Hexadimethrine chloride according to the INCI (CTFA) nomenclature.

(9) Polyquaternary ammonium polymers comprising units of formula (XIII):

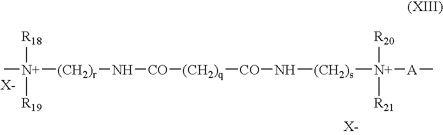

wherein:

R₁₈, R₁₉, R₂₀ and R₂₁, which may be identical or different, are chosen from a hydrogen atom, a methyl, ethyl, propyl, β-hydroxyethyl, β-hydroxypropyl, and —CH₂CH₂(OCH₂CH₂)ₚOH radical,

p is chosen from 0 and an integer ranging from 1 to 6, with the proviso that R₁₈, R₁₉, R₂₀ and R₂₁ do not simultaneously represent a hydrogen atom,

r and s, which may be identical or different, are integers ranging from 1 to 6,

q is chosen from 0 and an integer ranging from 1 to 34,

X⁻ is an anion such as a halide, and

A is a dihalide radical or for example is —CH₂—CH₂—O—CH₂—CH₂—.

Such compounds are described, for instance, in patent application EP-A-122 324.

Among these products, mention may be made, for example, of the products MIRAPOL® A 15, MIRAPOL® AD1, MIRAPOL® AZ1, and MIRAPOL® 175 sold by the company Miranol.

(10) Quaternary polymers of vinylpyrrolidone and of vinylimidazole, such as polyquaternium-11, polyquaternium-16 and polyquaternium-44, for example the products sold under the names LUVIQUAT® FC 905, FC 550, FC 370 and LUVIQUAT® Care by the company BASF.

(11) Polyamines, such as POLYQUART® H sold by Cognis, referenced under the name Polyethylene Glycol (15) Tallow Polyamine in the CTFA dictionary.

(12) Crosslinked or noncrosslinked methacryloyloxy(C₁-C₄)alkyltri(C₁-C₄)alkyl-ammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, for example methylenebisacrylamide. A crosslinked acrylamide/methacryloyloxyethyltrimethyl-ammonium chloride copolymer (20/80 by weight) in the form of a dispersion containing 50% by weight of the scopolymer in mineral oil may be used for example. This dispersion is sold under the name SALCARE® SC 92 by the company Ciba. A crosslinked methacryloyloxyethyltrimethylammonium chloride homopolymer containing about 50% by weight of the homopolymer in mineral oil or in a liquid ester can also be used. These dispersions are sold under the names SALCARE® SC 95 and SALCARE® SC 96 by the company Ciba.

Other cationic polymers that may be used are cationic proteins or cationic protein hydrolysates, polyalkyleneimines, such as polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present disclosure, the cellulose ethers comprising quaternary ammonium groups such as the products sold under the name JR 400 by the company Amerchol, cationic cyclopolymers, such as the homopolymers of diallyldimethylammonium salt and the copolymers of diallyldimethylammonium salt and of acrylamide, such as the chlorides, sold under the names MERQUAT® 100, MERQUAT® 550 and MERQUAT® S by the company Nalco, and guar gums modified with 2,3-epoxypropyltrimethylammonium chloride, for example sold under the name JAGUAR C13S by the company Rhodia, and mixtures thereof, may, for example be used.

The at least one cationic polymer may be present in the composition in an amount ranging from 0.001% to 20% by weight, for example from 0.01% to 10% by weight, and as a further example from 0.05% to 3% by weight relative to the total weight of the final composition.

The composition disclosed herein may have a pH ranging from 3 to 12 and for example from 4 to 8. The pH may be adjusted to the desired value conventionally, by adding a base (organic or mineral base) to the composition, for example aqueous ammonia or a primary, secondary or tertiary (poly)amine, such as monoethanolamine, diethanolamine, triethanolamine, isopropanolamine or 1,3-propanediamine, or alternatively by adding an acid, for example a carboxylic acid, such as citric acid.

The cosmetically acceptable medium may comprise water or a mixture of water and a cosmetically acceptable solvent such as a C₁-C₄ lower alcohol, such as ethanol, isopropanol, tert-butanol or n-butanol; alkylene glycols, for example propylene glycol, and glycol ethers. For example, the composition may comprise from 50% to 95% by weight of water relative to the total weight of the composition.

The composition disclosed herein may also contain various adjuvants usually used in cosmetics, such as fragrances, preserving agents, foam synergists, sequestering agents, foam stabilizers, propellents, dyes, polymers, such as amphoteric polymers, antidandruff agents, ceramides, pseudoceramides, vitamins and provitamins, hydroxy acids, acidifying and basifying agents, plant, mineral and animal oils, synthetic oils such as polyisobutenes and polydecenes, non-polyoxyethylenated fatty acid esters and nacreous agents, and other adjuvants depending on the intended use.

In addition to the combination defined above, the composition disclosed herein may also contain viscosity regulators such as electrolytes, or thickeners. Mention may be made, for example, of sodium chloride, sodium xylenesulphonate, cellulose derivatives, such as, for example, carboxymethylcellulose, hydroxypropylcellulose or hydroxyethylcellulose, guar gum, hydroxypropyl guar gums, amphiphilic polymers comprising at least one fatty chain, scleroglucans, xanthan gums, fatty acid alkanolamides, alkyl ether carboxylic acid alkanolamides optionally oxyethylenated with up to 5 mol of ethylene oxide, such as the product sold under the name AMINOL A15 by the company Chem Y, crosslinked polyacrylic acids and crosslinked acrylic acid/C₁₀-C₃₀ alkyl acrylate copolymers. These viscosity regulators are used in the composition in an amount up to 10% by weight relative to the total weight of the composition.

The composition disclosed herein may also contain up to 5% of nacreous agents and/or opacifiers that are well known, such as coated or uncoated titanium oxides, sodium or magnesium palmitate, sodium or magnesium stearate or hydroxystearate, fatty-chain acyl derivatives such as ethylene glycol or polyethylene glycol monostearate or distearate, fatty-chain ethers (for example of C8-C40), such as distearyl ether or 1-(hexadecyloxy)-2-octadecanol, cyclodextrins, and fatty alcohols, such as cetyl alcohol, stearyl alcohol and behenyl alcohol.

Also disclosed herein is the use of the composition defined above as a shampoo.

The composition disclosed herein may also be in the form of rinse-out or leave-in conditioners, permanent-waving, hair-relaxing, dyeing or bleaching compositions, or alternatively in the form of rinse-out compositions to be applied before or after dyeing, bleaching, permanent-waving or relaxing the hair or alternatively between the two steps of a permanent-waving or hair-relaxing operation.

Also disclosed herein is a process for washing keratin materials, comprising applying to these materials at least one composition as disclosed herein, followed by rinsing the treated materials after an optional leave-in time.

The examples that follow are intended to illustrate the invention without, however, being limiting in nature.

### EXAMPLES 1 to 3

The shampoo compositions below were prepared:

| Composition | Example 1 | Example 2 | Example 3 |
|---|---|---|---|
| Sodium lauryl ether sulphate | 7 g AM | 7 g AM | 7 g AM |
| (70/30 C12/C14) containing | | | |
| 2.2 mol of ethylene oxide | | | |
| Sodium cocoyl | 3.8 g AM | 3.8 g AM | 3.2 g AM |
| amphohydroxy- | | | |
| propylsulphonate, | | | |
| as an aqueous solution | | | |
| containing 32% active | | | |
| material, sold under the | | | |
| name MIRANOL CSE by the | | | |
| company Rhodia | | | |
| Oxyethylenated (200 OE) | 7 g AM | 7 g AM | 0.5 g AM |
| palm glyceride and | | | |
| oxyethylenated (7 OE) | | | |
| coconut glyceride | | | |
| mixture, an aqueous | | | |
| 70% suspension | | | |
| Oxyethylenated (30 OE) | 1 g | — | — |
| coconut mono/diglycerides | | | |
| Sorbitan monolaurate 4 OE | — | 0.5 g | 7 g |
| Sorbitan monolaurate 20 OE | — | 2.5 g | 1 g |
| Hydroxyethylcellulose | 0.25 g | 0.25 g | 0.25 g |
| crosslinked with | | | |
| epichlorohydrin and | | | |
| quaternized with | | | |
| trimethylamine, sold under | | | |
| the name JR 400 by | | | |
| the company Amerchol | | | |
| Preserving agents | qs | qs | qs |
| Citric acid qs | pH 7 | pH 5.3 | pH 7 |
| Demineralized water qs | 100 g | 100 g | 100 g |

Hair washed with these compositions was smooth and disentangled easily.

### EXAMPLE 4

The shampoo composition below was prepared:

| | |
|---|---|
| Sodium lauryl ether sulphate (70/30 | 4.5 g AM |
| C12/C14) containing 2.2 mol of ethylene | |
| oxide | |
| Sodium cocoyl amphohydroxy- | 2.3 g AM |
| propylsulphonate, as an aqueous solution | |
| containing 32% active material, sold | |
| under the name MIRANOL CSE by the | |
| company Rhodia | |
| Oxyethylenated (200 OE) palm glyceride | 4.8 g AM |
| and oxyethylenated (7 OE) coconut | |
| glyceride mixture, an aqueous 70% | |
| suspension | |
| Hydroxyethylcellulose crosslinked with | 0.2 g |
| epichlorohydrin and quaternized with | |
| trimethylamine, sold under the name | |
| JR 400 by the company Amerchol | |
| Preserving agents | qs |
| Citric acid qs | pH 5.3 |
| Demineralized water qs | 100 g |

What is claimed is:

1. A cosmetic composition comprising, in a cosmetically acceptable medium,
at least one amphoteric surfactant chosen from alkylamphohydroxyalkyl-sulphonate compounds and salts thereof, and
at least one polyethylene glycol ester or ether comprising at least one C₈-C₄₀ hydrophobic group.

2. The composition according to claim 1, wherein the at least one amphoteric surfactant is chosen from alkylamphohydroxyalkylsulphonate compounds of formula (I) below: 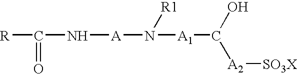
wherein:
R is chosen from saturated and unsaturated, linear and branched hydrocarbon-based radicals comprising from 5 to 29 carbon atoms,
R₁ is a C₁-C₄ hydroxyalkyl radical,
A, A₁, and A₂, which may be identical or different, are chosen from linear and branched C₁-C₁₀ alkylene radicals, and
X is chosen from a hydrogen atom, a mineral cation, and an organic cation.

3. The composition according to claim 2, wherein R1 is a hydroxyethyl radical.

4. The composition according to claim 2, wherein A, A₁, and A₂, which may be identical or different, are chosen from linear and branched C₁-C₃ divalent alkylene radicals,

5. The composition according to claim 2, wherein R is chosen from mono- and polyunsaturated alkyl and alkenyl radicals comprising from 5 to 29 carbon atoms.

6. The composition according to claim 5, wherein R is chosen from mono- and polyunsaturated alkyl and alkenyl radicals comprising from 7 to 22 carbon atoms.

7. The composition according to claim 6, wherein R is chosen from mono- and polyunsaturated alkyl and alkenyl radicals comprising from 9 to 17 carbon atoms.

8. The composition according to claim 2, wherein A and A₂ are —CH₂CH₂—.

9. The composition according to claim 2, wherein A₁ is —CH₂—.

10. The composition according to claim 2, wherein at least one of the mineral cation and the organic cation is chosen from an alkali metal, an alkaline-earth metal, an NH₄⁺ ion, and ammonium ions derived from basic amino acids or from amino alcohols.

11. The composition according to claim 10, wherein the alkali metal is chosen from Na⁺ and K⁺.

12. The composition according to claim 10, wherein the alkaline-earth metal is chosen from Mg²⁺ and Ca²⁺.

13. The composition according to claim 1, wherein the at least one amphoteric surfactant is chosen from cocoyl amphohydroxypropyl sulphonate salts and palmitoyl amphohydroxypropyl sulphonate salts.

14. The composition according to claim 1, wherein the at least one amphoteric surfactant is present in the composition in an amount ranging from 0.1% to 30% by weight relative to the total weight of the composition.

15. The composition according to claim 14, wherein the at least one amphoteric surfactant is present in the composition in an amount ranging from 1.5% to 20% by weight relative to the total weight of the composition.

16. The composition according to claim 1, wherein the at least one polyethylene glycol ester or ether comprising at least one C₈-C₄₀ hydrophobic group is chosen from compounds of formulae (II) and (III) below:
R₂—(O—CH₂—CH₂)n—OR₃ (II)
R₂—(O—CH₂—CH₂)m—OH (III)
wherein:
R₂ is chosen from linear and branched, saturated and unsaturated alkyl, acyl, alkylaryl, and aralkyl groups comprising from 8 to 40 carbon atoms, a glyceryl group, a (C₈-C₄₀)alkylglyceryl group, an acyl group derived from lanolin oil or lanolin fat, coconut oil, jojoba oil, castor oil, sweet almond oil, groundnut oil, wheatgerm oil, rice bran oil, flax oil, apricot kernel oil, walnut oil, palm oil, pistachio oil, sesame seed oil, rapeseed oil, cade oil, maize oil, peach nut oil, poppy oil, pine oil, soybean oil, avocado oil, sunflower seed oil, hazelnut oil, olive oil, grapeseed oil, safflower oil, shea butter, and babassu oil,
R₃ is chosen from a hydrogen atom, a linear and branched, saturated and unsaturated alkyl and acyl groups comprising from 1 to 40 carbon atoms, and
n and m are integers ranging from 3 to 350.

17. The composition according to claim 16, wherein R₂ is an acyl group comprising from 12 to 20 carbon atoms.

18. The composition according to claim 16, wherein R₃ is an acyl group comprising from 12 to 20 carbon atoms.

19. The composition according to claim 16, wherein n is an integer ranging from 100 to 300.

20. The composition according to claim 1, wherein the at least one polyethylene glycol ester or ether comprising at least one C₈-C₄₀ hydrophobic group is chosen from the compound of formula (IV) below: 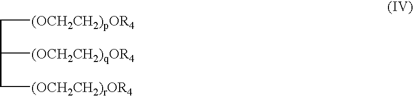
wherein R₄ is chosen from a hydrogen atom, and linear and branched, saturated and unsaturated alkyl, acyl, alkylaryl and aralkyl groups comprising from 8 to 40 carbon atoms,
p, q and r, which may be identical or different, range from 0 to 350,
the sum p+q+r ranges from 5 to 350, and
with the proviso that at least one of the groups R₄ is other than a hydrogen atom.

21. The composition according to claim 20, wherein the groups R₄ are acyl groups derived from plant oils.

22. The composition according to claim 21, wherein the plant oils are chosen from coconut oil, jojoba oil, castor oil, sweet almond oil, groundnut oil, wheatgerm oil, rice bran oil, flax oil, apricot kernel oil, walnut oil, palm oil, pistachio oil, sesame seed oil, rapeseed oil, cade oil, maize oil, peach nut oil, poppy oil, pine oil, soybean oil, avocado oil, sunflower seed oil, hazelnut oil, olive oil, grapeseed oil, safflower oil, shea butter, and babassu oil.

23. The composition according to claim 20, wherein the at least one polyethylene glycol ester or ether comprising at least one C₈-C₄₀ hydrophobic group is chosen from palm oil glycerides oxyethylenated with 200 mol of ethylene oxide, coconut oil mono-, di- and triglycerides oxyethylenated with 30 mol of ethylene oxide, and coconut oil mono-, di- and triglycerides oxyethylenated with 7 mol of ethylene oxide.

24. The composition according to claim 1, wherein the at least one polyethylene glycol ester or ether comprising at least one C₈-C₄₀ hydrophobic group is present in the composition in an amount ranging from 0.1% to 20% by weight relative to the total weight of the composition.

25. The composition according to claim 1, wherein the at least one polyethylene glycol ester or ether comprising at least one C₈-C₄₀ hydrophobic group is present in the composition in an amount ranging from 2% to 15% by weight relative to the total weight of the composition.

26. The composition according to claim 1, further comprising at least one additional surfactant chosen from nonionic, amphoteric, and zwitterionic surfactants other than the at least one amphoteric surfactant of claim 1.

27. The composition according to claim 26, wherein the at least one additional surfactant is present in an amount ranging from 0.1% to 60% by weight relative to the total weight of the composition.

28. The composition according to claim 26, wherein the combined total amount of surfactants ranges from 3.5% to 55% by weight relative to the total weight of the composition.

29. The composition according to claim 28, wherein the combined total amount of surfactants ranges from 7% to 30% by weight relative to the total weight of the composition.

30. The composition according to claim 1, further comprising at least one cationic surfactant.

31. The composition according to claim 1, further comprising at least one silicone.

32. The composition according to claim 1, further comprising at least one polymer chosen from at least one cationic polymer and at least one amphoteric polymer.

33. The composition according to claim 32, wherein the at least one polymer is chosen from those containing units comprising primary, secondary, tertiary and/or quaternary amine groups that either may form part of the main polymer chain or may be borne by a side substituent directly attached thereto.

34. The composition according to claim 32, wherein the at least one cationic polymer is chosen from cellulose ethers comprising quaternary ammonium groups, homopolymers of diallyldimethylammonium salt, copolymers of diallyidimethylammonium salt and of acrylamide, and guar gums modified with 2,3-epoxypropyltrimethylammonium chloride.

35. The composition according to claim 32, wherein the at least one polymer is present in an amount ranging from 0.001% to 20% by weight relative to the total weight of the composition.

36. The composition according to claim 35, wherein the at least one polymer is present in an amount ranging from 0.01% to 10% by weight relative to the total weight of the composition.

37. The composition according to claim 1, further comprising at least one viscosity regulator chosen from electrolytes and thickeners.

38. The composition according to claim 37, wherein the electrolytes are chosen from sodium chloride, and wherein the thickeners are chosen from cellulose derivatives, guar gum, hydroxypropyl guar gums, scleroglucans, xanthan gum, and amphiphilic polymers comprising at least one fatty chain.

39. The composition according to claim 1, further comprising at least one adjuvant chosen from fragrances, preserving agents, foam synergists, sequestering agents, foam stabilizers, propellants, dyes, antidandruff agents, ceramides, vitamins, provitamins, hydroxy acids, acidifying agents, basifying agents, plant, mineral oils, animal oils, synthetic oils, non-polyoxyethylenated fatty acid esters, pseudoceramides, nacreous agents, and opacifiers.

40. The composition according to claim 39, wherein the synthetic oils are chosen from polyisobutenes and polydecenes.

41. The composition according to claim 39, wherein the nacreous agents and opacifiers are chosen from coated titanium oxides, uncoated titanium oxides, sodium palmitate, magnesium palmitate, sodium stearate, sodium hydroxystearate, magnesium stearate, magnesium hydroxystearate, fatty-chain acyl derivatives, C₈-C₄₀ fatty-chain ethers, cyclodextrins, and fatty alcohols.

42. The composition according to claim 41, wherein the fatty-chain acyl derivatives are chosen from ethylene glycol, polyethylene glycol monostearate, and polyethylene glycol distearate.

43. The composition according to claim 41, wherein the C₈-C₄₀ fatty-chain ethers are chosen from distearyl ether and 1-(hexadecyloxy)-2-octadecanol.

44. The composition according to claim 41, wherein the fatty alcohols are chosen from cetyl alcohol, stearyl alcohol, and behenyl alcohol.

45. A method for treating keratin fibers comprising applying to keratin fibers a composition comprising, in a cosmetically acceptable medium, at least one amphoteric surfactant chosen from alkylamphohydroxyalkylsulphonate compounds and salts thereof, and at least one polyethylene glycol ester or ether comprising at least one C₈-C₄₀ hydrophobic group.

46. A method for washing keratin fibers comprising applying to keratin fibers a shampoo composition comprising, in a cosmetically acceptable medium, at least one amphoteric surfactant chosen from alkylamphohydroxyalkylsulphonate compounds and salts thereof, and at least one polyethylene glycol ester or ether comprising at least one C₈-C₄₀ hydrophobic group.

47. A method for imparting to hair at least one property chosen from improved disentangling and improved smoothness, comprising applying to hair a composition comprising, in a cosmetically acceptable medium, at least one amphoteric surfactant chosen from alkylamphohydroxyalkylsulphonate compounds and salts thereof, and at least one polyethylene glycol ester or ether comprising at least one C₈-C₄₀ hydrophobic group.

48. A method for imparting to hair at least one property chosen from volume, lightness, softness, flexibility or manageability, comprising applying to hair a cosmetic composition comprising at least one amphoteric surfactant chosen from alkylamphohydroxyalkylsulphonate compounds and salts thereof and at least one polyethylene glycol ester or ether comprising at least one C₈-C₄₀ hydrophobic group.

49. A method for washing keratin materials, comprising applying to the materials a composition comprising, in a cosmetically acceptable medium, at least one amphoteric surfactant chosen from alkylamphohydroxyalkylsulphonate compounds and salts thereof, and at least one polyethylene glycol ester or ether comprising at least one C₈-C₄₀ hydrophobic group, and
rinsing the materials after an optional leave-in time.
